(19) United States
(12) Patent Application Publication
(10) Pub. No.: US 2011/0092881 A1
(43) Pub. Date: Apr. 21, 2011

(54) IONTOPHORETIC DEVICE WITH CONTACT SENSOR

(75) Inventors: Emma Amelia Durand, Jamestown, RI (US); Michael K. Jordan, North Smithfield, RI (US)

(73) Assignee: Isis Biopolymer Inc., Providence, RI (US)

(21) Appl. No.: 12/903,924

(22) Filed: Oct. 13, 2010

Related U.S. Application Data
(63) Continuation-in-part of application No. 12/776,346, filed on May 7, 2010.
(60) Provisional application No. 61/176,719, filed on May 8, 2009.
(60) Provisional application No. 61/304,013, filed on Feb. 12, 2010.
(60) Provisional application No. 61/302,658, filed on Feb. 9, 2010.

(51) Int. Cl. A61N 1/30

(52) U.S. Cl. 604/20

(57) ABSTRACT

The present application discloses an iontophoretic device with a contact electrode for detecting placement of the device and/or characteristics of the wearer's tissue.

The present application is a continuation-in-part of U.S. patent application Ser. No. 12/776,346, the entirety of which is incorporated herein by reference.

### FIELD OF THE INVENTION

The present invention relates to an iontophoretic device, and particularly to various improvements to the same.

### BACKGROUND OF THE INVENTION

Iontophoretic devices are known in the art. They are placed on a patient's skin and use charged electrodes to drive charged drug ions from a drug reservoir and into the patient's skin tissue.

Two major shortcomings of current iontophoretic technology are (1) passive transfer of drug ions from the drug reservoir into the patient's skin tissue when the device is inactive, and (2) irritation to the patient's skin tissue because its impedance is used as an element of the circuit between two oppositely charged electrodes of the device. This has traditionally occurred because prior devices have used at least two spaced apart electrodes with the circuit being completed through the wearer's tissue, typically the skin.

It has been known to provide a contact sensor on an iontophoretic device for detecting operational placement of the device on the wearer's tissue. See, e.g., U.S. Pat. No. 5,991,655, the entirety of which is incorporated herein by reference.

The present application provides improvements in sensing proper placement and/or operational parameters of an iontophoretic device.

### SUMMARY OF THE INVENTION

One aspect of the present invention provides an iontophoretic device for delivering a drug into the tissue of a wearer. The device comprises: a base; a drug reservoir containing a supply of charged drug ions; a driving electrode positioned above the drug reservoir; a counterelectrode positioned below the drug reservoir opposite the driving electrode; and a control circuit including a power source. The control circuit is coupled to the driving electrode and the counterelectrode and operable in a driving mode for applying a potential to the driving electrode of the same polarity as the charge of the charged drug ions and a potential of opposite polarity to the counterelectrode so as to drive the charged drug ions towards the tissue of the wearer. A contact electrode is on a bottom of the body for contact with the user's tissue. The contact electrode is coupled to the controller for application of a potential of a polarity opposite the counterelectrode. The controller is configured to detect current flow through the wearer's tissue between the contact electrode and the counterelectrode.

Another aspect of the invention relates to an iontophoretic device for delivering a drug into the tissue of a wearer. The device comprises: a base; a drug reservoir containing a supply of charged drug ions; a plurality of electrodes including a driving electrode positioned above the drug reservoir and a counterelectrode; and a control circuit including a power source. The control circuit is coupled to the driving electrode and the counterelectrode and operable in a driving mode for applying a potential to the driving electrode of the same polarity as the charge of the charged drug ions and a potential of opposite polarity to the counterelectrode so as to drive the charged drug ions towards the tissue of the wearer. A contact electrode is on a bottom of the body for contact with the user's tissue. The contact electrode is coupled to the controller for application of a potential. The controller is configured to detect current flow through the wearer's tissue between the contact electrode and at least one of the plurality of electrodes. The control circuit includes a sensor for detecting a parameter of the current flow through the wearer's tissue between the contact electrode and the at least one of the plurality of electrodes. The control circuit is configured to adjust a parameter of current flow and/or the potential difference between the driving electrode and the counterelectrode based on sensor's detection of the density of current flow through the wearer's tissue between the contact electrode and the at least one of the plurality of electrodes.

Other objects, features, and advantages of the present invention will become apparent from the following detailed description, the accompanying drawings, and the appended claims

### BRIEF DESCRIPTION OF THE DRAWINGS

FIG. 1 is an exploded cross-sectional view of a device constructed in accordance with the present invention, with top and bottom views also included;

FIG. 2 is a cross-sectional view showing the electrodes and drug reservoir in isolation;

FIG. 3 is an exaggerated cross-section of an alternative embodiment;

FIG. 4 is an example of a control circuit for the embodiment of FIG. 3.

FIG. 5 is another example of a control circuit for the embodiment of FIG. 3;

FIG. 6 is a top view of another embodiment of the invention;

FIG. 7 is an exploded view of the embodiment of FIG. 6; and

FIG. 8 is a bottom view of the embodiment of FIG. 8

### DETAILED DESCRIPTION OF THE ILLUSTRATED EMBODIMENT(S) OF THE INVENTION

The Figures illustrate non-limiting embodiments of an iontophoretic device 10. The device 10 may have any construction and configuration, and the illustrated embodiment is not intended to be limiting.

The basic principles of iontophoretic devices are well-known, and reference may be made to U.S. Patent Publication No. 2009/0048556 and U.S. Publication No. 2009/0299267 A1 for teachings in this regard, the entirety of which are incorporated herein. Reference may also be made to the above-incorporated '346 application.

The device 10 comprises a base 12. The base 12 is preferably a flexible structure, such as a foam or plastic, and is designed to conform to the body of the patient and lie adjacent the skin. The base 12 has a drug reservoir opening 14 formed therethrough, which contains a drug reservoir 16. The base 12 may have any construction or configuration, and the illustrated embodiment is not intended to be limiting.

The drug reservoir 16 contains a supply of charged drug ions, which may be elemental ions (i.e., the ionic form of an element), molecular ions (i.e., the ionic form of a molecule), complexed ions (i.e., ions of a weakly bonded group of elements/molecules/ions referred to as a complex). In the illustrated embodiment, the reservoir comprises a gel, such as a hydrogel. The drug may be solvated in a solution in charged ionic form along with the polymer for the gel, and upon curing the polymer cross-links and the charged drug ions are stored in the gel. For example, a salt of the drug may be dissolved in the solution, thus providing drug ions with mobility within the ionically conductive solution/gel. The methods by which such drug reservoirs are formed are known and need not be detailed herein. For example, the drug reservoir 16 may simply be the gel as shown, or it may have a more complex structure, such as a partitioned reservoir with an internal membrane for separating and managing ion mobility. The drug reservoir may have any construction or configuration, and the illustrated embodiment is not intended to be limiting.

The term drug may include any bioactive agent, such as pharmaceuticals, vitamins, treatments, elements, etc., and is not limited to just those drugs subject to regulatory approval. As such, the term drug should be interpreted as meaning any agent having a biological effect on the wearer that is transdermally delivered by the device.

A barrier layer 18 is positioned below the drug reservoir so as to be positioned between the drug reservoir and the tissue of the wearer. The barrier layer has the same configuration as or is larger than the drug reservoir 16 and its opening 14 in terms of area. That is, the barrier layer 18 covers the entire drug reservoir 16, thus maintaining its position between the drug reservoir 16 and the wearer's skin. The barrier layer 18 is configured to prevent essentially prevent passive transport of the charged drug molecules therethrough.

In the illustrated embodiment, the barrier layer 18 is a mesh. The mesh may be coated with an electroconductive material, such as, for example, Ag, AgCl, or carbon. The coating may vary based on the specific drug molecule, delivery rate, and other requirements. The mesh may have any pore size, such as, for example, between 7 and 100 microns. The pore size may also vary depending on the specific drug molecule, delivery rate, and other requirements.

The barrier layer 18 in the illustrated embodiment is formed as part of a layer 20 of the device 10, which is adhered or otherwise bonded to the bottom surface of the base 12. This layer 20 is not necessary, nor is it necessary to form the barrier layer 18 as part of the layer 20.

An adhesive layer 28 may be coated about the peripheral edge of the layer 20. The adhesive layer is preferably a high tack adhesive for firmly bonding the device 10 against the patient's skin. By extending the adhesive to the peripheral edge of the retainer 20 and device 10, the adhesive serves to discourage lifting or peeling of the edges of the device 10, thus maintaining it securely fastened to the skin. Other suitable attachment means may be used to secure the device to a patient, such as tape, straps, etc.

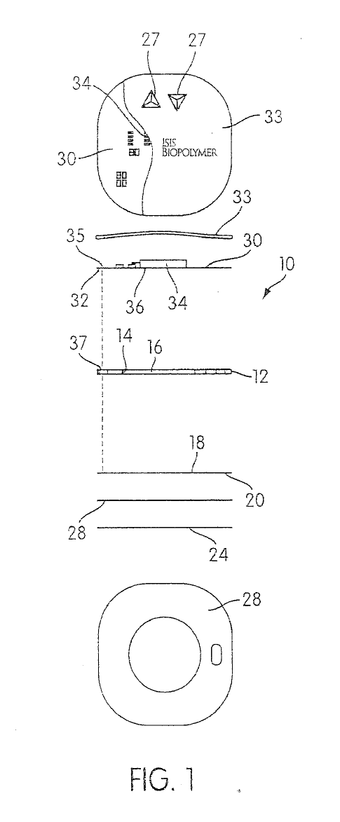

An optional release liner 24 covers the entirety of the bottom surface of the device 10. That is, the release liner 24 covers both the adhesive 28 and may also cover the area of the drug reservoir 16. The release liner 24 may be paper, plastic or another material, and the upper side of the release liner 24 has a release material, such as silicone or wax, so it can be peeled off to expose the adhesive layer 28 and the drug reservoir 16. The release liner 24 is omitted in the bottom view of FIG. 1 so the drug reservoir area can be seen.

Turning to the portions of the device 10 above the base 12 and the drug reservoir 16, the device 10 further comprises a circuit layer 30. The circuit layer is preferably formed of a dielectric (i.e., electrically insulative) substrate 32, such as a flexible non-conductive polymer substrate that can flex to conform to various parts of the patient's body. The upper surface of the substrate 32 includes circuitry, formed preferably as a printed circuitry deposited by polymer thick film coating. That coating technique is disclosed in the above-referenced US Patent Publication 2009/0048556, which may be referred to for its teachings in that regard.

The upper surface of the substrate 32 also includes a power source in the form of a battery. Preferably, the battery may be of the printed type, also taught in US Patent Publication No. 2009/0048556, although any type of battery/power source may be used. A microprocessor 34 is also mounted to the upper surface of the substrate 32, and is coupled to the circuitry and power source for controlling the delivery of electrical power.

Collectively, the circuitry, microprocessor and the power source may be considered a control circuit that controls the application of potentials to the electrodes used in the device 10, which are discussed below. The microprocessor may be omitted, and switches may be used in the control circuit for controlling current flow/direction and the application of the various potentials to the electrodes. Thus, the term control circuit is a structural term that encompasses any circuit coupled to the electrodes for applying potentials thereto, including circuits with or without a microprocessor, integrated circuits, and/or switch-operated circuits.

An optional cover layer 33 is affixed to the substrate 32 to cover and protect the components provided on substrate 32. The cover layer 33 is partially shown in the top view of FIG. 1 to show its relationship to the components, and typically covers all the components.

The device 10 may include one or more activation switches coupled to the control circuit, such as shown at 27. For example, there may be two switches: an on switch for activating the driving mode (as discussed below), and an off switch for stopping the driving mode or activating a forced inactive mode (as discussed below). The switches may be of any type, including membrane switches, button switches, contact switches, piezoelectric, or any other type.

A through-hole (not shown) is formed through the substrate 32 and enables the circuitry on the upper surface of the substrate 32 to be connected to a driving electrode 36 provided on the bottom surface of the substrate 32. The driving electrode 36 is also referred to in the art as a donor electrode. Preferably, the driving electrode 36 is also printed on the bottom surface of the substrate 32 using the polymer thick film coating technique mentioned above. A printed lead may extend from the through-hole to the electrode 36, depending on the placement of the electrode 36 relative to the through-hole. A flexible conductive ink material such as silver or carbon, may fill the through-hole and connect the circuit on the upper surface to the driving electrode 36 or its lead, and also prevents the infiltration of water up through the through-hole to the circuit. This couples the driving electrode 36 to the control circuitry, thus enabling the power source to apply a potential thereto. Any other suitable way of coupling the driving electrode 36 to the control circuit may also be used.

The driving electrode 36 is positioned above the drug reservoir 16 opposite the barrier layer 18. The driving electrode 36 preferably has the same size and configuration as the drug reservoir 16 and its opening in terms of area, thus enabling the potential applied to the driving electrode 36 to be exposed to the entire drug reservoir 16. During operation in a driving mode, the control circuit applies a potential to the driving electrode 36 of the same polarity as the charge of the charged drug ions so as to drive the charged drug ions towards and into the tissue of the wearer. That is, if the drug is in the form of a positively charged ion, the driving electrode 36 will have a positive charge applied to it. Because charges of the same polarity repel, the positively charged drug ions will be repelled away from the driving electrode 36 and driven towards the tissue of the wearer's skin for permeation into the skin. Conversely, if the drug ion is in the form of a negatively charged ion, the driving electrode 36 will have a negative charge applied to it, thus similarly repelling and driving the drug ions.

The barrier layer 18 is configured to permit the charged drug ions to be actively transported therethrough in the driving mode via the potential applied to the driving electrode 36. That is, the barrier layer 18 may be constructed such that it normally prevents passive transport of the charged drug molecules, but allows the active driven transport by the electromotive force of the driving electrode 36 to occur.

In the illustrated embodiment, the barrier layer 18 is formed of an electroconductive material and is also coupled to the control circuit. To establish the connection, aligned through-holes 35, 37 may be formed through the substrate 32 and the base 12, thus allowing for a lead to couple the barrier layer 18 to the control circuit on the upper surface of substrate 32. A lead may also be formed, such as by polymer thick film printing, on layer 20 depending on the relative placements of the barrier layer 18 and the through-holes 35, 37. For example, the lead may be provided on the upper surface of layer 20 and extend laterally from the through hole 37 to the barrier layer 18, which are spaced laterally apart as shown in FIG. 2. A conductive epoxy 39 may be used as mentioned above to establish the connection between the control circuit and barrier layer 18, and to also prevent water migration. However, any other ways of connecting the control circuit to the barrier layer 18 may also be used, including wires, leads, contacts or a z-axis material. The relative sizing of elements in the cross-section of FIG. 2 is somewhat exaggerated to facilitate a better understanding, and different sizes and dimensions may be used.

The control circuit is configured to apply a potential of opposite polarity to the barrier layer 18 so that the barrier layer functions as a counterelectrode. That is, the potential of the driving electrode 18 is applied from one terminal of the power source, whereas the potential of the barrier layer 18 functioning as a counterelectrode is applied from the opposite terminal of the power source. The term counterelectrode specifically refers to and means the electrode that is counter or opposite in charge to the driving electrode 36 and is provided for purposes of completing the iontophoretic circuit between the connections to the opposing terminals of the power source. The microprocessor of the control circuit may be configured to control the application of potentials to both electrodes, and various circuit elements may be used to determine the potential and current density applied to each electrode to ensure proper delivery of the drug molecules.

Preferably, the gel of the drug reservoir 16 is electroconductive, thus completing the circuit comprising the driving electrode 36 and barrier layer/counterelectrode 18. The gel preferably has sufficiently high resistance to maintain a sufficiently high potential difference between the electrodes. Alternatively, rather than rely on the drug reservoir for electroconductively coupling the electrodes, a resistor or other element with a level of resistance may be used to enable the current flow between the electrodes while maintaining an adequate potential difference between the electrodes.

As an example, lidocaine contained in a water-based gel can be delivered using a current density of 0.2 mA/cm² (assuming the driving electrode 36 and counterelectrode/barrier layer 18 have the same area).

Because the barrier layer 18 when used as a counterelectrode will have the opposite polarity as the charged drug molecules in the driving mode, this may enhance the transport of the drug ions. This is because the charged drug molecules will be both repelled away from the driving electrode 36, and attracted towards the barrier layer 18. This may beneficially increase the rate of drug transport achieved per unit power, since both electrodes are contributing to drug transport in the same direction towards the tissue of the wearer.

This construction with the barrier layer 18 serving as the counter-electrode is also advantageous in terms of device size and patient comfort. With prior art devices, typically a counterelectrode is spaced apart laterally from the driving electrode, and the circuit is completed through the impedance or resistance of the patient's skin tissue. While sometimes the counterelectrode can be used with a drug reservoir having drug molecules of a charge opposite the drug in the other reservoir, in many instances only a single drug is being delivered, and thus a “passive” drug-free reservoir is used. In either situation, the device can be irritating because the patient's skin tissue is effectively part of the circuitry, and thus there are practical limits to the power that can be applied to the electrodes. For example, the prior art devices are known to cause burning and “tattooing” (the presence of visible marking) of patient skin. This is a significant drawback of prior art designs. Moreover, if only a single drug is being delivered, a significant portion of the overall area of the device 10 is dedicated to the non-drug delivering passive electrode and reservoir. Even if the counterelectrode is used for purposes of delivering a second drug, it still has the patient discomfort/irritation issue and also is limited to drug ions of the same polarity as its potential (i.e., of charge that is opposite the charge of the drug ions in the other reservoir), thus limiting the potential range of applications that can justify the larger size. With the illustrated embodiment, such an issue is eliminated because there is no laterally spaced counterelectrode that needs to complete the circuit through the wearer's tissue—the circuit is completed within the device with the current flowing between the electrodes across a resistance within the device.

Another advantage is that the gel of the reservoir can maintain a stable conductivity, whereas the conductivity or impedance of skin tissue can vary depending on various conditions, including pH, perspiration, etc. Thus, the device 10 where the barrier layer 18 serves as the counterelectrode eliminates that problem, as the conductivity of the drug reservoir is essentially independent of skin conditions.

Without being limited to a specific mechanism of action, it is believed that the use of the driving electrode and counterelectrode on opposing sides of the drug reservoir creates a high concentration of drug ions at the counterelectrode, which facilitates osmotic transport/permeation of the drug ions into the patient's skin/tissue. Where the counterelectrode is a mesh or permeable membrane, for example, and placed directly against the patient's tissue/skin, this creates intimate contact to further improve such permeation. With prior art devices having the electrodes spaced laterally from one another, the skin itself is part of the “circuit,” as discussed above, and the potential difference is between the electrodes through the skin, which is the primary force in the delivery of the drug. In contrast, the use of the driving electrode and counterelectrode on opposing sides of the drug reservoir overcomes the shortcomings of these prior art devices, while still enabling a sufficient amount of drug to be delivered. Indeed, it is possible to use even higher power for the delivery of the drug ion with the opposing driving and counter electrodes because skin is not part of the iontophoretic circuit. This theory of operation is not intended to be limiting. It may be possible in some embodiments that a potential difference could be established between the counterelectrode and the wearer's tissue, which may play a role in drug delivery, but it is believed that any such potential difference would be minor when compared to the controlled larger potential difference between the electrodes within the device itself.

In some embodiments, when it is desired to further minimize the ability of drug ions to passively transport across the barrier layer 18, its polarity may be reversed when the drug is not being delivered. That is, the control circuit may be configured to operate in a “forced inactive” mode and reverse the counterelectrode polarity so that it has the same charge as the drug ions, thus repelling the drug ions away from the patient's skin tissue. Similarly, the polarity of the driving electrode 36 may also be reversed by the control circuit in the forced inactive mode, thus enhancing the repulsive effect of the barrier layer 18 by attracting the drug molecules towards the driving electrode 36 (and hence away from the wearer's skin tissue) by virtue of having the opposite charge as the drug ions. This may be done at a very low power to preserve battery life. This mode of operation may be referred to as forced inactive mode, and the control circuit is configured to be switched to this forced inactive mode to apply these potentials. The term “forced inactive” is used to denote this mode because the device 10 is inactive for delivering the drug, but electrical force is being used to enhance the drug delivery prevention.

In some embodiments, the reversed potentials in this forced inactive mode may be applied to the driving electrode and counterelectrode at predetermined intervals, such as in pulses in accordance with a predetermined duty cycle. This is done to minimize the energy drawn in the inactive mode. Advantageously, the two electrodes when charged will drive the molecules towards to driving electrode 36 and away from the counterelectrode 18 and the wearer's skin tissue. Because passive migration back towards the skin will happen rather slowly, the reversed potentials can be pulsed or intermittently applied to offset that passive migration. Thus, a continuous current draw may not be necessary in the inactive mode. In some embodiments, the counterelectrode may be used irrespective of whether it functions as a barrier layer. That is, the counterelectrode located opposite the driving electrode 36 with the drug reservoir 16 therebetween may be used to minimize or eliminate the flow of current into the user's skin. In such an embodiment, the counterelectrode need not cover the entire bottom surface of the drug reservoir. For example, the counterelectrode may have an annular configuration. Any other construction or configuration may be used.

The device 10 may also have an optional contact sensor to determine that the device is placed properly in contact with a user's tissue. For example, a relatively small contact electrode 42 may be used. This contact electrode 42 may be formed in the same way on layer 20 as the counterelectrode 18. The contact electrode 42 may be coupled to the circuitry on the upper surface of substrate 32 using through-hole connections similarly to the counterelectrode 18. Specifically, aligned through hole 44 and 46 are formed in substrate 32 and base 12, respectively, and filed with an electroconductive material 48, such as an epoxy. The control circuitry on the upper surface of substrate 32 can detect if counterelectrode 18 and contact electrode 42 are in contact with the user's tissue using various techniques. For example, the contact electrode 42 could be set with a polarity opposite the counterelectrode 18 so that establishment of a current flow therebetween can be detected. This may be done by intermittent sampling to prevent continuous current draw, and/or at a very low current flow to prevent tissue irritation. More specific details concerning the contact electrode 42 and its operation will be provided below.

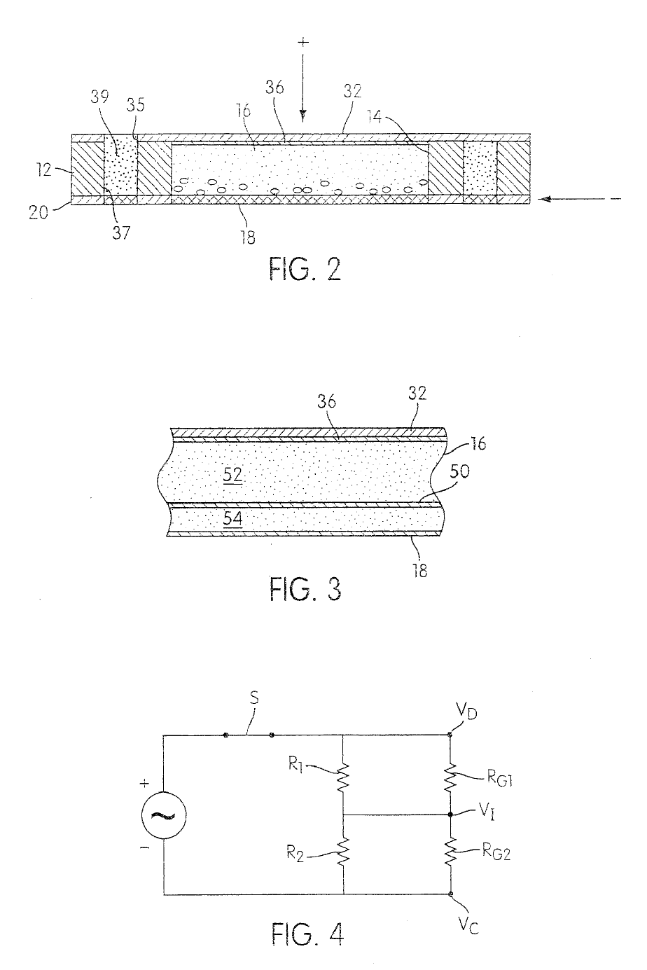

FIG. 3 is a schematic view of another embodiment of the present invention. Similar components are used, and thus the same reference numbers will be used for components common to this embodiment and the prior one. The device in its entirety is not shown, and only the electrodes and drug reservoir are illustrated, as the device may otherwise be generally the same.

FIG. 3 shows the driving electrode 36, the counterelectrode 18 (which need not be a barrier layer), the drug reservoir 16, and an intermediate electrode 50. The intermediate electrode 50 is positioned between the driving electrode 36 and the counterelectrode 18. Preferably, the intermediate electrode 50 separates the drug reservoir 16 into two portions: a first portion 52 located between the driving electrode 36 and the intermediate electrode 50, and a second portion 54 located between the intermediate electrode 50 and the counterelectrode 18. The intermediate electrode 50 may be disposed in the reservoir 16 in any manner. For example, where the drug reservoir 16 is a gel, the intermediate electrode 50 may be placed in position and set in place as the gel cures. Also, the first and second portions 52, 54 may be separately formed and placed on opposing sides of the intermediate electrode 50. In some embodiments, multiple intermediate electrodes may be used.

The control circuit may be coupled to the intermediate electrode 50 and be operable in the driving mode to apply a potential to the intermediate electrode 50 that is between the potentials applied to the driving electrode 36 and the counterelectrode 18 so as to drive the charged drug ions from the first portion 52 of the drug reservoir 16 into the second portion 54 of the drug reservoir 16 and drive the charged drug ions in the second portion 54 of the drug reservoir 16 towards the tissue of the wearer. That is, the potential difference between the driving electrode 36 and the intermediate electrode 50 is such that, for the drug ions in the drug reservoir's first portion 52, the driving electrode 36 has the same polarity as the charged drug ions and the intermediate electrode 50 has the opposite polarity, thus driving the charged drug ions in the drug reservoir's first portion 52 towards the second portion 54. Similarly, the potential difference between the intermediate electrode 50 and the counterelectrode 18 is such that, for the drug ions in the drug reservoir's second portion 54, the intermediate electrode 50 has the same polarity as the charged drug ions and the counterelectrode 18 has the opposite or counter polarity, thus driving the drug ions from the drug reservoir's second portion 54 towards the wearer's tissue in the same manner as described above. (It should be noted that “polarity” is relative to an opposite or counter electrode, and thus it is correct to state that the intermediate electrode 50 has one polarity (e.g., positive) when compared to the driving electrode 36 and an opposite polarity (e.g., negative) when compared to the counterelectrode 18.)

Without being limited to a specific mechanism of action, it is believed that the drug ions migrate from the first portion 52 of the drug reservoir 16 to the second portion 54 by a “push-pull” action. Specifically, the drug ions in the first portion 52 are driven to the intermediate electrode 50, which is essentially the interface between the first and second reservoir portions 52, 54, by the potential difference between the driving and intermediate electrodes 36, 50. At this interface, the potential difference between the intermediate electrode 50 and the counterelectrode 18 further drives the drug ions away from the intermediate electrode 50 and towards the counterelectrode 18 and patient tissue. Thus, at the interface provided by the intermediate electrode 50, the drug ion migration or transport may be described as being “pushed” towards and then “pulled” away from the intermediate electrode 50 by the potential differences relative to the driving electrode 36 and counterelectrode 18, respectively.

The resistances between the driving electrode 36/intermediate electrode 50 and the intermediate electrode 50/counterelectrode 18 pairs through which current flow is established may be provided by the material of the drug reservoir 16, such as an electroconductive gel, or other resistors, as discussed above. This also enables current flow from the driving electrode 36 to the counterelectrode 18.

Preferably, the spacing between the counterelectrode 18 and the intermediate electrode 50 is less than the spacing between the intermediate electrode 50 and the driving electrode 36. This provides various advantages in both the driving mode, a passive mode, and a forced inactive mode (if used).

The rate at which a drug ion is transported in an ionically conductive drug reservoir is a function of the potential difference between the electrodes on opposing sides of the reservoir, as well as the distance between the electrodes. Thus, from a power efficiency standpoint, closely spaced electrodes are more efficient. However, narrowing the gap between the electrodes also reduces the volume of drug reservoir therebetween (and hence the amount of drug ions stored therein). These are competing factors in the design of a typical iontophoretic device: power efficiency relative to drug delivery rate versus overall volume of drug stored.

With the presence of the intermediate electrode 50, it can be placed closely to the counterelectrode 18 to increase its contribution per unit power to drug delivery rate from the drug reservoir second portion 54, while a larger volume of drug can be stored in the larger first portion 52 between the intermediate electrode 50 and the further spaced driving electrode 36.

Also, the intermediate electrode 50 may be a membrane that reduces or prevents passive transport of the drug ions from the drug reservoir first portion 52 to the second portion 54. This limits the amount of drug ions available for passive absorption into the patient's tissue when the device 10 is not being operated (i.e., the passive mode) to the much smaller amount present in the second portion 54. Even if the intermediate electrode membrane allows some passive transport of the drug ions into the second portion 54, this still acts as an upper limit on the long term passive absorption rate. The counterelectrode 18 may also be constructed as a barrier layer, such as a membrane, as discussed above, to further restrict or prevent passive absorption of the drug ions. Alternatively, the counterelectrode 18 may be open mesh that does not substantially interfere with drug transport.

When either the intermediate electrode 50 or counterelectrode 18 formed as a membrane, it may be formed for any membrane material, including, but not limited to mesh or cloth materials that are metallic or non-metallic, and which may be coated or printed with conductive ink. Preferably the intermediate electrode membrane 50 is hydrophobic to further reduce the transport of drug ions therethrough.

Preferably, the spacing between the intermediate electrode 50 and the counterelectrode 18 is less than or equal to 50% of the spacing between the intermediate electrode 50 and the driving electrode 36. More preferably, that value is less than or equal to 30%, 20%, or 10%. These values are not limiting.

In an embodiment, similarly to the embodiment discussed above, the control circuit is switchable to a forced inactive mode. In this forced inactive mode, the control circuit may at least apply a potential to the counterelectrode 18 of the same polarity as the charge of the charged drug ions and a potential of opposite polarity to the driving electrode 36, thus repelling the drug ions away from the tissue of the wearer. That is, because of the attractive nature of the driving electrode's 36 potential and the repulsive nature of the counterelectrode's 18 potential, the drug ions are encouraged to migrate away from the counterelectrode 18 and the wearer's tissue towards the driving electrode 36.

As an option, the control circuit may also be configured such that in the forced inactive mode the control circuit applies a potential to the intermediate electrode 50 that is between the potentials applied to the driving electrode 36 and the counterelectrode 18. Thus, within the drug reservoir second portion 54, the drug ions are repelled away from the counterelectrode 18 and the wearer's tissue, and attracted towards the intermediate electrode 50; and within the drug reservoir's first portion 52 the drug ions are repelled away from the intermediate electrode 50 and attracted towards the driving electrode 36. Thus, the drug ions are repelled away from the tissue of the wearer and from the drug reservoir second portion 54 to the first portion 52. The same “push-pull” effect may occur at the intermediate electrode 50 as described above, albeit in reverse.

This advantageously uses electrical power to prevent or reduce passive absorption of the drug ions into the wearer's tissue. Preferably, the relatively closer spacing between the counterelectrode 18 and intermediate electrode 50 enhances the rate at which the drug ions are transported within the drug reservoir second portion 54, and the larger spacing between the driving electrode 36 and the intermediate electrode 50 provides increased volume for storage of the drug ions distal from the patient's tissue.

In another embodiment, the control circuit in the inactive mode may apply potentials only to the counterelectrode 18 and the intermediate electrode 50. That is, the control circuit applies a potential to the counterelectrode 18 of the same polarity as the charge of the charged drug ions, and a potential of opposite polarity to the intermediate electrode 50. This repels the drug ions away from the counterelectrode 18 and the tissue of the wearer, and attracts the drug ions to the intermediate electrode 50. Because this will create a high concentration of drug ions at the intermediate electrode 50, some of the drug ions may passively migrate to the first portion 52 of the drug reservoir 16 by osmosis.

In yet another embodiment, the control circuit in the inactive mode may apply potentials only to the intermediate electrode 50 and the driving electrode 36. That is, the control circuit applies a potential to the intermediate electrode 50 of the same polarity as the charge of the drug ions and a potential of opposite polarity to the driving electrode 36. This repels the drug ions away from the intermediate electrode 50, and attracts the drug ions to the driving electrode 36. This prevents or reduces transport of the drug ions into the drug reservoir's second portion 54, thus limiting the amount of drug available for passive absorption into the wearer's tissue. Also, because this will result in low or zero concentration of drug ions in the area of the first drug reservoir portion 52 adjacent the intermediate electrode 50, it is possible (but not necessary) that some drug ions will passively migrate from the second portion 54 to the first portion 52 due to osmosis and the concentration gradient.

In any variation of the forced inactive mode, the respective potentials applied to the electrodes (i.e., either all three electrodes, the driving electrode/counterelectrode pair, the driving electrode/intermediate electrode pair, or the intermediate electrode/counterelectrode pair) may be applied in predetermined intervals, as discussed above.

Although a microprocessor is preferred for precise control of the potentials applied to the electrodes 18, 36, 50, it may be omitted and the control may be provided by basic circuit elements as well.

For example, FIG. 4 shows the basic driving circuitry for a control circuit with no forced inactive mode. Nodes 18, 36, and 50 represent the counterelectrode, the driving electrode, and intermediate electrode, respectively. Resistors R_{G1} and R_{G2} represent the respective resistances of the gel drug reservoir portions between those electrodes (the G standing for gel, and the 1 and 2 standing for the first and second portions 52, 54 respectively). Resistors R₁ and R₂ constitute a voltage divider for dividing the voltage difference to set the intermediate electrode 50 at an intermediate potential. Switch S, shown in a closed position, connects the power source in the closed position to power the circuit (thus establishing a driving mode) and disconnects the power source in the open position (thus establishing a passive mode).

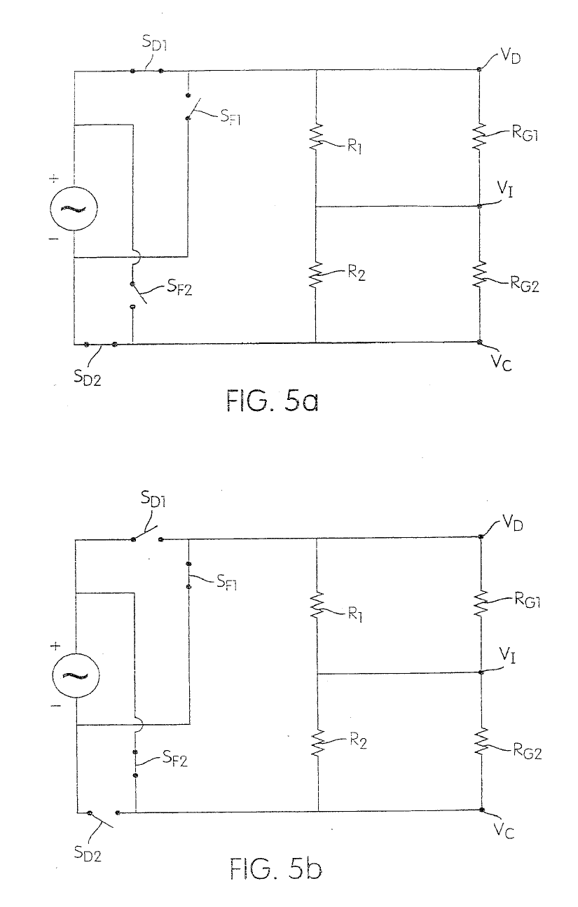

FIGS. 5a and 5b show a circuit similar to FIG. 4, except two pairs of switches S_{D1}/S_{D2} and S_{F1} and S_{F2} are provided. Switches S_{D1}/S_{D2} when closed couple the terminals of the power source in one polarity configuration to establish the driving mode (and the switches S_{F1} and S_{F2} are open), as shown in FIG. 5a. In FIG. 5b, the switch positions are reversed, with switches S_{D1} and S_{D2} open and switches S_{F1} and S_{F2} closed, thus reversing the polarity configuration and establishing the forced inactive mode. In particular, this forced inactive mode has potentials applied to all three electrodes.

In FIGS. 4, 5a, and 5b, V_{D}, V_{I}, and V_{C} schematically denote the nodes at which the driving electrode 36, the intermediate electrode 50, and the counterelectrode 18 are located, and their voltages V_{D}, V_{I}, V_{C} are controlled as described above. Although the example circuits shown are configured for driving positively charged drug ions, the power supply voltage applied can be reversed for driving negatively charged drug ions.

These circuit diagrams are examples only and are not intended to be limiting. Any circuit arrangements may be used.

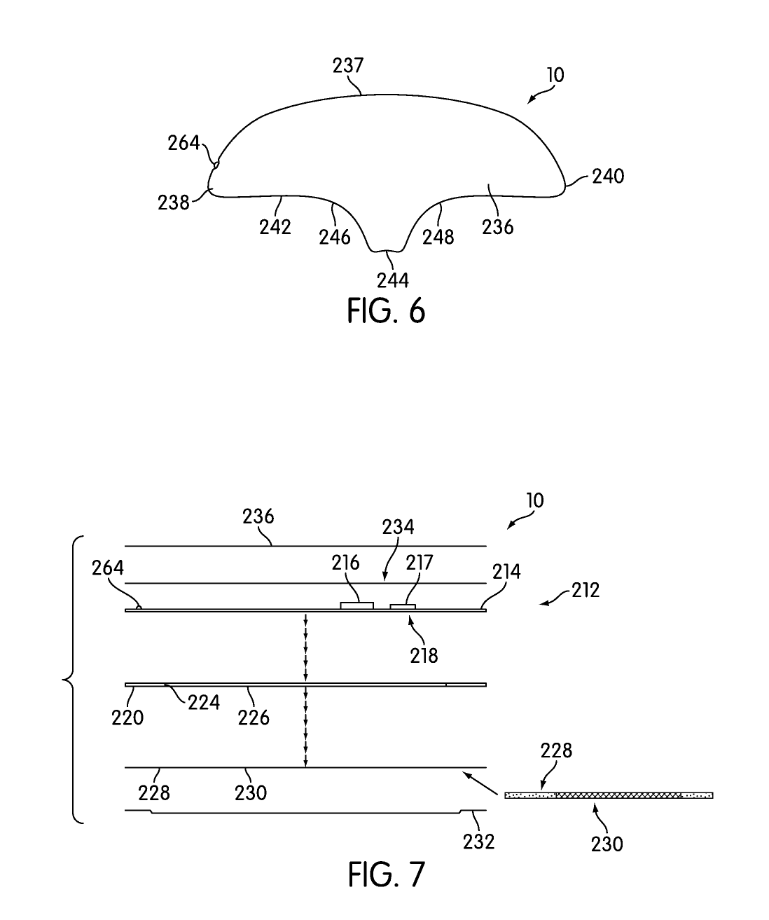

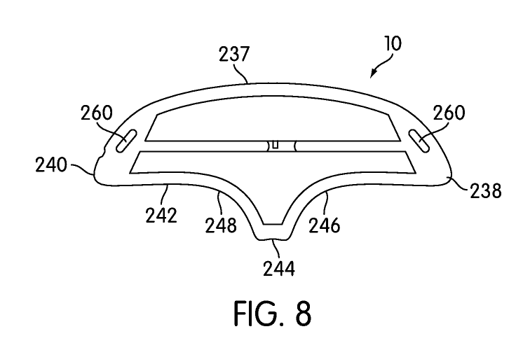

FIGS. 6-8 illustrate another embodiment of the device 10, includes a body 212. FIG. 7 is shown an exploded view, and uses the same type of through-hole connections discussed above, or other suitable connections. FIGS. 6 and 8 show top and bottom views, respectively. The illustrated embodiment is designed particularly for placement on the brow/forehead region of a person, as will be discussed in detail below. However, certain aspects of the device 10 may be used in devices designed for other uses, and the brow/forehead configuration should not be regarded as limiting with respect to other aspects useable in other contexts. Moreover, a brow/forehead configuration may have other constructions, and need not have all the features described herein, and may include other features not explicitly described.

The body 212 includes a base 214, which is in the form of a flexible circuit board substrate. The base 214 is formed of a dielectric material and includes an upper surface on which a controller 216 is provided. The controller 216 includes circuitry that may be printed on the upper surface of the base 214 and may constitute any type of control circuit as mentioned above. A power source in the form of a battery 217 is also provided to provide electrical current for operating the device 10, as will be described below.

A driving electrode 218 is provided on the bottom surface of the base 214. The driving electrode 218 may be connected to the controller 216 and/or its circuitry on the upper surface of the base 214 via a through-hole connection, for example. The driving electrode 218 may be provided in any way, such as is discussed above, and may be printed on the bottom surface of the base 214, such as by a printed coating of electroconductive ink. The ink may be, for example, printed silver or silver chloride.

As used herein, the terms upper and lower are used to denote the directions facing away and towards the user's skin tissue, respectively. These terms do not denote a specific orientation of usage, but rather are used in reference to the position of use in reference to a patient's skin surface. These terms are used for convenience only.

The body 212 also includes a drug reservoir layer 220. The drug reservoir layer 220 includes a drug reservoir opening 224 that receives a drug reservoir 226. The drug reservoir layer 220 has the same peripheral shape and dimensions as the base 214, and is secured to the bottom surface of the base 214 in any suitable manner, such as with an adhesive. The drug reservoir layer 220 surrounds and contains the drug reservoir 26 within the device. The drug reservoir layer 220 is preferably made from a flexible material, including but not limited to a foam, such as a closed cell foam.

The drug reservoir 226 preferably has the same peripheral shape and dimensions as the driving electrode 218 to facilitate iontophoretic delivery of drugs from the drug reservoir 226. The drug reservoir 226 contains charged drug ions for delivery into the user's skin tissue. The charged drug ions are mobile within the reservoir, meaning they can migrate within the reservoir, such as is caused by the iontophoretic delivery described below. The reservoir may be an aqueous gel (i.e., a water-based gel), or any other type of medium for containing the charged drug ions and permitting mobility thereof. The drug may be dissolved within the gel into ionic form. The ions may include any ionic form, including elemental ions, molecular ions, or ions of a complex (i.e., ions of a weaker bonded coordinated atoms/molecule). Further details are provided above and/or in the incorporated applications

By way of example, the drug ions contained in the reservoir 226 may include, but not are not limited to salycylic and hyaluronic acid.

A counterelectrode support layer 228 provided beneath the drug reservoir layer 220. The counterelectrode support layer 228 has the same peripheral shape and dimensions as the base 214 and drug reservoir layer 220, and is also made from a flexible material. The outer edge portions of the counterelectrode support layer 228 are secured to the bottom surface of the drug reservoir layer 220. The central portion of the counterelectrode support layer is formed as a counterelectrode 230, which preferably has the same peripheral dimensions and shape as the drug reservoir 226 and the driving electrode 218. The function of the counterelectrode 230 will be described below. Preferably, at least the counterelectrode region of the layer 228 is formed of a permeable mesh or membrane material, including but not limited to including, but not limited to mesh or cloth materials that are metallic or non-metallic. The entire layer 228 may be made of such material, or it may comprise an outer peripheral region formed of a different material to which the counterelectrode 230 is attached. The counterelectrode 230 may be formed in any manner, such as by printing or coating with an electroconductive ink, such as silver or silver chloride.

An optional underlayer 232 may also be provided and secured to the bottom surface of the counterelectrode support layer 228. This optional underlayer 232 has an open area adjacent the counterelectrode 230, and preferably of the same peripheral size and dimensions. This open area permits the drug ions to pass through the counterelectrode 230 to the user's skin, as will be described below.

An adhesive is coated on the underlayer 232 (or the counterelectrode support layer 28 if the underlayer 232 is omitted). This adhesive serves to secure the device 10 to the patient's skin. Other ways of securement may be used.

Above the base 214, the body 212 also includes a spacer layer 234. This spacer layer 234 extends about the periphery of the base 214 and is secured to the base in any suitable manner, such as by using an adhesive. The spacer layer 214 maintains a spacing between the upper cover 236 and the base 214, and helps to protect the components on the base. The spacer layer 214 may be made from any suitable material, such as a closed cell foam, and is preferably flexible. The cover layer 236 is secured to the top surface of the spacer layer 234, and covers the base 214 and the components thereon. The cover layer 236 may be made of any suitable material, such as plastic, cloth, a membrane, of a thin closed-cell foam.

As can be appreciated from the foregoing, the use of flexible materials for the various layers allows the device 10 to flex to conform to various contours of the user's body. The use of printed circuitry and printed electrodes facilitates such flexibility as well.

The general configuration of the illustrated embodiment is designed for use as a brow/forehead device. Specifically, the device is elongated in the lateral direction so as to cover a significant lateral extent of the forehead. The hairline conforming edge 237 of the device 10 is curved convexly so as to fit beneath and along the hairline, with the curvature proceeding downwardly towards the outer lateral ends 238, 240 of the device 10. The brow conforming edge 242 has a central extension 244 for positioning between the user's eye brows, and concave curves 246, 248 extending laterally outwardly therefrom towards the opposing lateral ends 238, 240 for extending over the eye brows. Preferably, the device 10 is symmetrical.

Preferably, the device 10 when adapted for brow/forehead may have the following dimensions. The lateral length between the opposing ends 240, 244 may be between 4 and 6 inches, and preferably is 5.225 inches. The vertical length (from the bottom edge of the extension 244 to the center of the hairline edge 37) may be between 1.5 and 3 inches, and preferably is 2.363 inches. The length of the extension 244 as measured from the inflection point for the curves 246, 248 may be between 0.5 and 0.75, and preferably is 0.6. The radius of curvature for the hairline edge 237 may be between 2 and 4, and is preferably 2.95. And the radius of curvature for the curves 246, 248 may be between 0.7 and 1, and is preferably 0.9. These values are examples only, and are not intended to be limiting. Moreover, as mentioned above, various aspects of the device 10 may be practiced for other applications, and the brow/forehead configuration in general is not intended to be limiting.

In use, the device 10 operates in a driving mode to transport the charged drug ions from the drug reservoir 226 into the user's skin tissue. The process used is iontophoresis, as discussed above. In the driving mode, the battery 217 applies a potential via the controller to the driving electrode 218 of the same polarity as the charge of the drug ions. Thus, if the drug ions have a positive charge, a potential of positive charge is applied to the driving electrode 218; and if the drug ions have negative charge, a potential of negative charge is applied to the driving electrode 218. Because like charges repel, the drug ions are repelled away from the driving electrode 218 and towards the user's skin tissue.

To facilitate this process, in the driving mode the battery 217 also applies a potential via the controller to the counterelectrode 230. The potential applied to the counterelectrode 230 is of the opposite polarity, meaning that it has a charge that is opposite the charge of the drug ions. This attracts the drug ions towards the counterelectrode 230 and the user's tissue, thus enhancing the repelling effect of the driving electrode 218 and contributing to the driving of the drug ions towards the user's skin tissue. Because the counterelectrode 230 is permeable, the drug ions can pass therethrough for absorption into the user's skin.

The specifics of how the driving electrode 218 and counterelectrode 230 positioned on opposing sides of the reservoir 226 operate are described in further detail above and in the '346 application incorporated above. The microprocessor of the control circuit may be configured to control the application of potentials to both electrodes, and various circuit elements may be used to determine the potential and current density applied to each electrode to ensure proper delivery of the drug molecules.

Because the counterelectrode 230 will have the opposite polarity as the charged drug molecules in the driving mode, this may enhance the transport of the drug ions. This is because the charged drug molecules will be both repelled away from the driving electrode 218, and attracted towards the counterelectrode 230. This may beneficially increase the rate of drug transport achieved per unit power, since both electrodes are contributing to drug transport in the same direction towards the tissue of the wearer.

This construction with the counterelectrode 230 is also advantageous in terms of device size and patient comfort, as is discussed above in more detail, as well as in the '346 application.

In some embodiments, when it is desired to further minimize the ability of drug ions to passively transport across the counterelectrode 230, its polarity may be reversed when the drug is not being delivered. That is, the control circuit may be configured to operate in a “forced inactive” mode as described above, as well as in the '346 application. The device may also be constructed using the intermediate electrode design described above and in the above-incorporated '346 application as well.

The device 10 also have an optional contact sensor to determine that the device is placed properly in contact with a user's tissue. For example, a relatively small contact electrode 260 on a bottom of the body 212 may be used. This contact electrode 260 may be formed in the same way on layer 228 as the counterelectrode 230. The contact electrode 260 may be coupled to the circuitry on the upper surface of substrate 232 using through-hole connections similarly to the counterelectrode 230. Specifically, the connections discussed above for contact electrode 42 may be used.

For either illustrated embodiment (or any other embodiment for that matter), the control circuitry on the upper surface of the respective substrate can detect if counterelectrode 18/230 and contact electrode 42/260 are in contact with the user's tissue using various techniques. For example, the contact electrode 42/60 could be set with a polarity opposite the counterelectrode so that establishment of a current flow therebetween can be detected. This may be done by intermittent sampling to prevent continuous current draw, and/or at a very low current flow to prevent tissue irritation.

A detection that the contact electrode 42/260 and the counterelectrode 18/230 are both in contact with the wearer's tissue via current being conducted therebetween many be used to initiate generation of the device to deliver the drug. Conversely, the lack of such detection may be used to cease operation of the device to deliver the drug.

Preferably, the current amperage between the contact electrode and the counterelectrode is substantially smaller than that used between the driving electrode and counterelectrode to avoid irritation of the patient's skin tissue. For example, the current flowing through the drug reservoir between the driving and counterelectrodes may be at or above 1 mA, while the current flowing through the patient's skin between counterelectrode and the contact electrode may be between 10 and 50 mA, and preferably 30 qA. The control circuit may perform the contact detection between the contact and counterelectrodes intermittently, at every 5-10 seconds for example. The drug delivery current may be ceased during the contact detection to avoid interference with the contact detection.

In an embodiment, during contact detection, the current amperage and potential difference between the counterelectrode and contact electrode may be used to determine the resistance between the electrodes. If the resistance determined is above a threshold, e.g. 5 mΩ, that may indicate that device is not in contact, and thus drug delivery is erased. If the resistance determined is at or below the threshold, that may indicate that the device is in contact, and thus drug delivery may begin or continue.

The control circuit may optionally include a sensor device in either embodiment, which may be logic residing in a controller or a hardwired module, for detecting the current density flowing between the counterelectrode 18/230 and the contact electrode 42/260. With the potentials applied to the counterelectrode 18/230 and the contact electrode 42/260 known, and thus the potential difference being known, the current density between those electrodes may fluctuate as a function of characteristics of the wearer's tissue. In particular, the hydration or moisture content of skin tissue may be a characteristic of interest, because that may effect the skin's ability to absorb and/or transport the drug being delivered. For example, a soluble drug may be more easily delivered into well-hydrated skin tissue, and thus less current density may be required between the driving electrode 36/218 and counterelectrode 18/230 to achieve a target drug delivery rate; conversely drier skin may require more current density to achieve the same target drug delivery rate. Thus, the sensor device in the control circuit may be used to adjust the current density between the driving electrode 36/218 and counterelectrode 18/230 to adjust the drug delivery rate based on the current density detected between the counterelectrode 218/230 and contact electrode 42/260. Similarly, in some embodiments the potential difference between driving electrode 36/218 and counterelectrode 18/230 may be adjusted to adjust the drug delivery rate. In some embodiments, both the potential difference and current density between driving electrode 36/218 and counterelectrode 18/230 may be adjusted to adjust the drug delivery rate.

As can be seen in the Figures, in the embodiment of FIGS. 6-8, two contact electrodes 260 are provided. The number of electrodes may vary, and this is not intended to be limiting. The two contact electrodes are both coupled to the control circuit in the same manner and used for the same purpose. The control circuit may average the signals from the two contact electrodes 260, or use one in preference with the other serving as a back-up. Or the circuit may alternate between sampling both at predetermined intervals to ensure that proper placement of the device on the wearer's tissue in both locations.

The ability of the device to adjust the current density used to deliver the drug ions may be used in conventional devices with laterally spaced apart electrodes. Thus, the contact electrodes could be used to detect a current between it and either of the plurality of electrodes in such a device.

In instances where current density is detected, another parameter of the current may be used. For example, the amperage independent of density may be detected. Thus, any parameter of the current flow may be detected. The device 10 may also include a light 264, such as an LED connected to the control circuit. The light may be positioned at an edge of the device 10 and be activated to be illuminated continuously or in a blinking manner to inform the user that the device 10 is operating for drug delivery. The control circuit may change the illumination pattern (i.e., go from continuous illumination to blinking) or cease illumination of the light 264 after a predetermined period of time to indicate to the user that the drug delivery is completed and that the device 10 may be removed. This is a beneficial feature for a device 10 design for a single use.

The foregoing embodiments have been provided solely for illustrating the structural and functional principles of the present invention and are not intended to be limiting. To the contrary, the present invention is intended to encompass all modifications, alterations, substitutions, and equivalents within the spirit and scope of the appended claims.

What is claimed:

1. An iontophoretic device for delivering a drug into the tissue of a wearer, the device comprising:
a base;
a drug reservoir containing a supply of charged drug ions;
a driving electrode positioned above the drug reservoir;
a counterelectrode positioned below the drug reservoir opposite the driving electrode;
a control circuit including a power source, the control circuit being coupled to the driving electrode and the counterelectrode and operable in a driving mode for applying a potential to the driving electrode of the same polarity as the charge of the charged drug ions and a potential of opposite polarity to the counterelectrode so as to drive the charged drug ions towards the tissue of the wearer; and
a contact electrode on a bottom of the body for contact with the user's tissue, the contact electrode being coupled to the controller for application of a potential of a polarity opposite the counterelectrode, the controller being configured to detect current flow through the wearer's tissue between the contact electrode and the counterelectrode.

2. An iontophoretic device according to claim 1, wherein the control circuit is configured to permit the operation in the driving mode upon detecting the current flow through the wearer's tissue between the contact electrode and the counterelectrode.

3. An iontophoretic device according to claim 1, wherein the control circuit includes a sensor for detecting a density of the current flow through the wearer's tissue between the contact electrode and the counterelectrode.

4. An iontophoretic device according to claim 3, wherein the control circuit is configured to adjust a density of current flow between the driving electrode and the counterelectrode based on sensor's detection of the density of current flow through the wearer's tissue between the contact electrode and the counterelectrode.

5. An iontophoretic device according to claim 1, further comprising a light indicator, said control circuit being coupled to the light indicator and configured to activate the light indicator to emit an activation signal in the driving mode.

6. An iontophoretic device according to claim 5, wherein said light indicator is an LED.

7. An iontophoretic device for delivering a drug into the tissue of a wearer, the device comprising:
a base;
a drug reservoir containing a supply of charged drug ions;
a plurality of electrodes including a driving electrode positioned above the drug reservoir and a counterelectrode;
a control circuit including a power source, the control circuit being coupled to the driving electrode and the counterelectrode and operable in a driving mode for applying a potential to the driving electrode of the same polarity as the charge of the charged drug ions and a potential of opposite polarity to the counterelectrode so as to drive the charged drug ions towards the tissue of the wearer; and
a contact electrode on a bottom of the body for contact with the user's tissue, the contact electrode being coupled to the controller for application of a potential, the controller being configured to detect current flow through the wearer's tissue between the contact electrode and at least one of the plurality of electrodes;
wherein the control circuit includes a sensor for detecting a parameter of the current flow through the wearer's tissue between the contact electrode and the at least one of the plurality of electrodes;
wherein the control circuit is configured to adjust a parameter of current flow and/or the potential difference between the driving electrode and the counterelectrode based on sensor's detection of the parameter of current flow through the wearer's tissue between the contact electrode and the at least one of the plurality of electrodes.

8. An iontophoretic device according to claim 7, wherein the parameter of the current flow detected by the sensor is current density.

9. An iontophoretic device according to claim 7, wherein the parameter of the current flow adjusted by the control circuit is the current density.

10. An iontophoretic device according to claim 8, wherein the parameter of the current flow adjusted by the control circuit is the current density.
